(19) United States
(12) Patent Application Publication
(10) Pub. No.: US 2011/0015649 A1
(43) Pub. Date: Jan. 20, 2011

(54) Surgical Guidance Utilizing Tissue Feedback

(75) Inventors: Mehran ANVARI, Hamilton (CA); John LYMER, Brampton (CA); Timothy FIELDING, Brampton (CA); Hon Bun YEUNG, Brampton (CA)

Correspondence Address: LICATA & TYRRELL P.C., 66 E. MAIN STREET, MARLTON, NJ, 08053, US

(73) Assignee: McMaster University, Hamilton (CA)

(21) Appl. No.: 12/842,462

(22) Filed: Jul. 23, 2010

Related U.S. Application Data
(63) Continuation of application No. PCT/CA2009/000076, filed on Jan. 23, 2009.
(60) Provisional application No. 61/006,655, filed on Jan. 25, 2008.

(51) Int. Cl. A61B 19/00

(52) U.S. Cl. 606/130

(57) ABSTRACT

A surgical system is for use with a surgical tool and a tissue characteristic sensor associated with the surgical tool. The system has an expected tissue characteristic for tissue on a predefined trajectory of the tool in a patient, and a controller to receive a sensed tissue characteristic from the tissue characteristic sensor, such sensed tissue characteristic associated with an actual trajectory of the tool. The controller compares the expected tissue characteristic for the expected location with the sensed tissue characteristic for the actual trajectory. A robot can be used to carry out automated surgical tasks and make adjustments based on differences between the expected characteristic and the sensed characteristic.

### CROSS-REFERENCE TO RELATED APPLICATIONS

This application is a continuation of PCT Patent Application PCT/CA2009/000076 filed Jan. 23, 2009 under title Surgical Guidance Utilizing Tissue Feedback which claims priority of U.S. Provisional Patent Application 61/006,655 filed Jan. 25, 2008 under title Multi-Purpose Robotic Operating System with Automated Feed Back. The contents of the above application is hereby incorporated by reference into the Modes of Carrying out the Invention hereof.

### TECHNICAL FIELD

The present application relates to guidance of surgical tools and to systems therefore. It also relates to automated robot performance of surgery and systems therefore.

### BACKGROUND

Many systems have been developed to assist with guiding surgeons use of tools in the performance of surgery. Ultimately, the tools used with such systems are under the control of the surgeon at all times.

Typically preoperative images are taken, the surgery is planned using the preoperative images, and the surgeon is provided with guidance information during surgery based on the estimated location of the tools in the images. Intraoperative images can be taken to update the image information.

Some systems have been considered that can perform surgical tasks using a robot acting in accordance with image guidance. The robot follows a preplanned path developed utilizing the images.

The time lag between actual time and when the last image was taken, image latency, is a significant concern in determination of the actual location of a tool at any time.

As well, for surgeon performed surgical tasks, image guidance information typically requires a surgeon to focus visually on the information and away from the specific location of surgical activity. Also, total radiation exposure to both medical personnel and the patient during image acquisition can have inherent dangers. Surgeon reaction time, feel, and manual control, whether direct or through intermediate tools, can limit the precision with which surgical tasks can be performed.

For surgical tasks performed by a robot under automated control utilizing image guidance, the robot follows a preplanned path, including any errors in the path. This may result in a negative outcome or requirement for manual intervention by the surgeon.

It is desirable to improve upon or provide alternatives for surgical guidance that address one or more of the above concerns or other concerns with the guidance of surgical tools.

### SUMMARY

In an aspect the invention provides a surgical system for use with a surgical tool and a tissue characteristic sensor associated with the surgical tool. The system includes an expected tissue characteristic for tissue on a predefined trajectory of the tool in a patient, and a controller to receive a sensed tissue characteristic from the tissue characteristic sensor, such sensed tissue characteristic associated with an actual trajectory of the tool, wherein the controller compares the expected tissue characteristic for the expected location with the sensed tissue characteristic for the actual trajectory.

The system may further include a display displaying information to an operator of the tool based on the compared expected tissue characteristic and sensed tissue characteristic.

The tool may be operated by an operator through manual operation of the tool.

The system may further include a robot for manipulating the tool, wherein the tool is operated by the operator through the operator manually operating the robot.

The system may include the tissue characteristic sensor. The system may include the surgical tool.

The system may include a robot for manipulating the tool under control of the controller, which control is based on the compared expected tissue characteristic and sensed tissue characteristic.

The tissue characteristic sensor may be a force sensor, the expected tissue characteristic may be a force characteristic of expected tissue on the predefined trajectory, and the sensed tissue characteristic may be a sensed force characteristic on the actual trajectory of the tool.

The system may include means for an operator to monitor robot performance while under the control of the controller. The system may include means for an operator to assume control away from the controller of the manipulation of the tool.

In another aspect the invention provides a method of using a surgical system. The method includes receiving at a controller within the surgical system from a tissue characteristic sensor a sensed tissue characteristic associated with an actual trajectory of a surgical tool, and comparing within the controller the expected tissue characteristic for the expected location with the sensed tissue characteristic for the actual trajectory.

The method may include displaying information on a display to an operator of the tool based on the compared expected tissue characteristic and sensed tissue characteristic.

The tool may be operated by an operator through manual operation of the tool.

The tool may be operated by the operator through the operator manually operating the robot for manipulating the tool.

The method may include sensing the tissue characteristic through the tissue characteristic sensor.

The method may include controlling a robot under control of the controller to manipulate the tool, which control is based on the compared expected tissue characteristic and sensed tissue characteristic.

The tissue characteristic sensor may be a force sensor, the expected tissue characteristic may be a force characteristic of expected tissue on the predefined trajectory, and the sensed tissue characteristic may be a sensed force characteristic on the actual trajectory of the tool.

Other aspects of the invention will be evident from the Modes of Carrying out the Invention and FIGS. provided herein.

### BRIEF DESCRIPTION OF THE DRAWINGS

Reference will now be made, by way of example, to the accompanying drawings which show example embodiments of the present application, and in which:

FIG. 1 is a block diagram of an example surgical system according to an embodiment of an aspect of the present invention;

FIG. 2 is a block diagram of a further example surgical system according to an embodiment of an aspect of the present invention;

FIG. 3 is a block diagram of another example surgical system according to an embodiment of an aspect of the present invention pedicle screw installation;

FIG. 4-7 are perspective views of an example embodiment of an aspect of the present invention in use to drill a pedicle screw hole in a vertebra;

FIG. 8 is a diagrammatic illustration of various examples forces sensed in some example embodiments of aspects of the present invention;

FIGS. 9A-9B shows fluoroscope images showing target region and tool (FIG. 8A-Lateral and FIG. 8B-A/P Image);

FIGS. 10A-10B show a patient mounted localizer array (PLA) from a distance and close-up;

FIG. 11 shows an imager in use from above;

FIG. 12 shows an imager in use from one side;

FIG. 13 is a perspective view of a robotic system at a surgical site;

FIG. 14 shows example start and end points Identified on the fluoroscope images of FIGS. 8A-8B;

FIG. 15 is a system interface diagram;

FIG. 16 illustrates a perspective view of an example manipulator arm of an example robot;

FIGS. 17A and 17B illustrates a back view and a side view of the example manipulator arm of FIG. 16;

FIG. 18 is a diagram of registration and tool tracking;

FIG. 19 is a diagram of an example system set up;

FIG. 20 is a diagram of an example robotic system at a surgical site;

FIG. 21 is a diagram of robotic system of FIG. 15 at the surgical site with user input of trajectory points;

FIG. 22 is a diagram of localization of points using two fluoroscopic images;

FIG. 23 is an example operation functional flow for a pedicle screw insertion; and

FIG. 24 is a block diagram of example system interfaces.

Similar reference numerals may be used in different figures to denote similar components.

### MODES FOR CARRYING OUT THE INVENTION

It is to be recognized that the examples described herein are only to be considered as examples, and any mention of requirements or needs, and key elements is to be interpreted in the context of the example only.

Throughout this description like components may be used with different embodiments. When describing embodiments with like components similar reference numerals may be used and the descriptive text may not be repeated; however, it is understood that the description of such components applies equally between the embodiments to the extent the context permits.

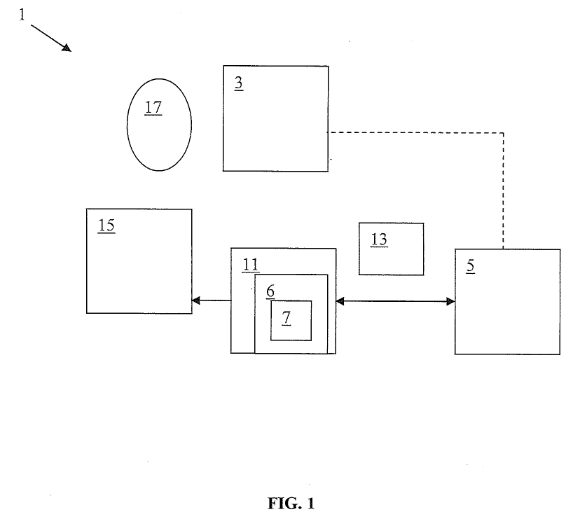

Referring to FIG. 1, a surgical system 1 is for use with a surgical tool 3 and a tissue characteristic sensor 5 associated with the surgical tool 3. The tool 3 and sensor 5 can be associated in many different ways. The sensor 5 may be on or a part of the tool 3. The sensor 5 and the tool 3 may be associated by tracking so that the relationship between the tool 3 and sensor 5 is known. In later embodiments, the sensor 5 may be part of a robot that manipulates the tool 3 such that relationship between the tool 3 and sensor 5 is known through the robot.

The system 1 stores in memory 6 an expected tissue characteristic 7 for tissue on a predefined trajectory of the tool 3. Expected tissue characteristics for surgical tasks can be stored as models in the memory 6 for use by the surgical system. A controller 11 receives a sensed tissue characteristic 13 from the tissue characteristic sensor 5. The sensed tissue characteristic 13 is associated with an actual trajectory of the tool 3. The controller 11 compares the expected tissue characteristic 7 for the expected location with the sensed tissue characteristic for the actual trajectory.

The predefined trajectory may be based on images as will be later discussed. Alternatively a surgeon may select a predefined trajectory through external viewing of a patient based on accumulated knowledge.

A display 15 displays information to an operator 17 of the tool 3 based on the compared expected tissue characteristic and sensed tissue characteristic. The tool 3 may be operated by an operator 17, for example a surgeon, through manual operation of the tool 3 with the operator of the tool 3 viewing the displayed information and manually operating the tool 3 accordingly.

Other interfaces, not shown, such as audible or tactile interfaces can be used to feedback information to the operator about the compared expected tissue characteristic and the sensed tissue characteristic. For example, a tactile increase in pressure to magnify the force on a handheld tool, or a robot operated tool (described below), may be used to provide information to an operator.

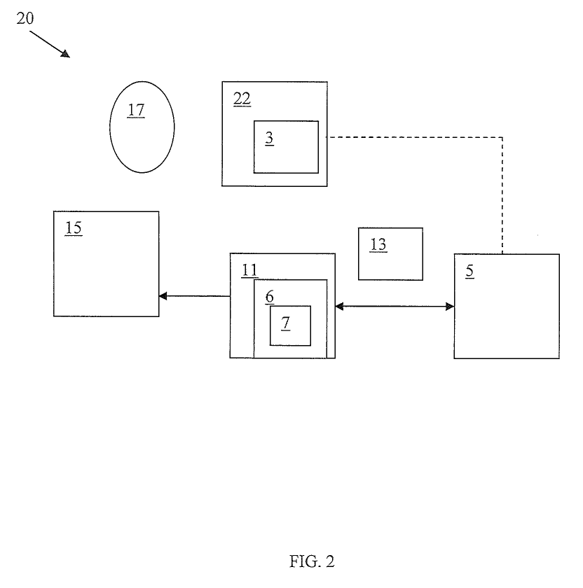

Referring to FIG. 2, a surgical system 20 is similar to system 1 and includes a robot 22 for manipulating the tool 3. it is understood that the tool 3 may take different forms on the different embodiments depending on how it is to be held and used. For example a hand held scalpel (as tool 3) may be different from a robot handheld scalpel (as tool 3), as will be known to those skilled in the art. The tool 3 is operated by the operator through the operator manually operating the robot.

The tissue characteristic sensor 5 may be supplied as part of the system 1 or may be provided separately. Similarly, the surgical tool 3 may be provided as part of the system 1 or may be provided separately.

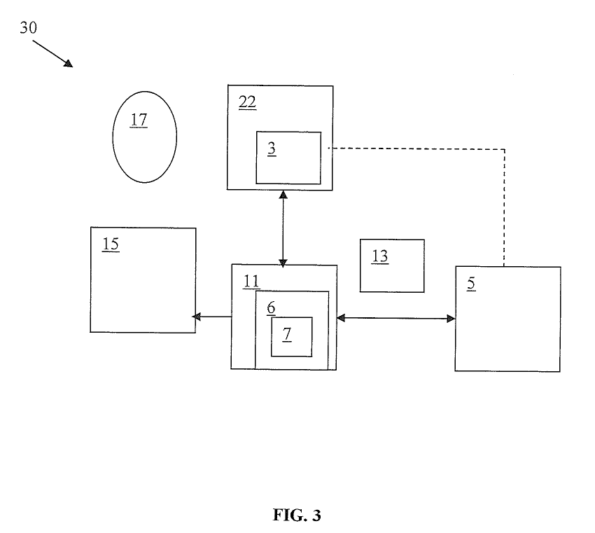

Referring to FIG. 3, a surgical system 30 is similar to the system 20. The robot 22 is under control of the controller, which control is based on the compared expected tissue characteristic and sensed tissue characteristic.

For robot 22 operation under control of the controller to perform automated surgical tasks in a pre-programmed manner it can be desirable to provide a number of redundant functions to enhance safety. For example, the surgical system 30 can incorporate the following safety features:

Internal health monitoring of the surgical system, for example signals within valid limits, processor watchdogs,

Redundant sensors to facilitate signal cross checking. For example robot 22 joints in the examples described herein have two position sensors that are checked against one another to ensure sensors are sending valid data. If these signals do not agree, an error is flagged and motion is halted,

Force feedback to limit applied tool forces,

Monitoring of patient position (for example, with a tracking system),

Monitoring of a robot end effector position with a tracking,

system to provide an independent observer check that position commands to the robot 22 are properly carried out,

“No go” zones defined within the surgical system, for example by software executed thereon, to limit the available workspace for the surgical task, which no go zones can include a combination of user defined and system defined zones (such as avoiding known objects or targets),

Visual indication of robot tool position on images to facilitate surgeon validation of registration,

Robot position feedback sensors are monitored against the commanded trajectory to ensure the robot is following the command within acceptable boundaries,

Tissue characteristic feedback that is typically tool specific, but can include sensors on the tools to detect specific types of tissues, Compensation for patient respiration using sensors and tracking,

Tracking of other active devices and imaging in the field to avoid collision,

Recognize and provide system response to tool malfunction, significant delay or sudden change in positioning outside the range that the system can adapt to,

A deadman switch,

External stop switch for manual cancellation of task at the operator's discretion, and

Providing a clear, distinct, multi-step process to enable robot motion with easily recognizable feedback to the operator.

Typically all of the available safety features designed into a given surgical system will be utilized for a given surgical task. For some surgical tasks, although a surgical system may permit the setting of user defined boundaries, whether or not a setting is entered will be at the discretion of the user. Feedback may be limited or not available for some tools.

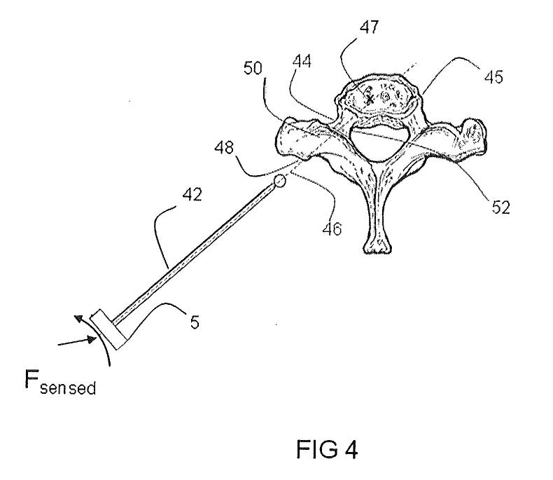

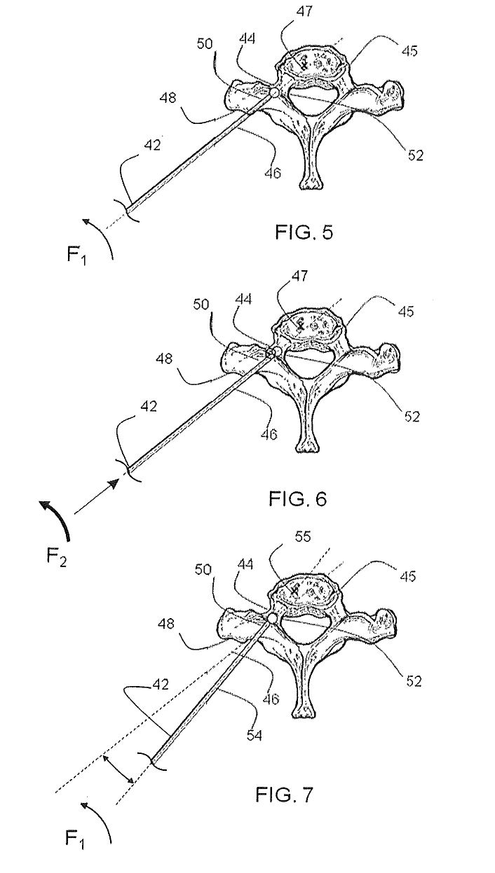

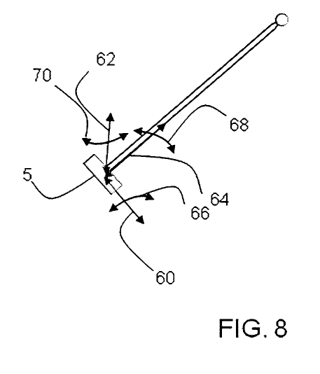

Referring to FIGS. 4-7, an example will be described utilizing an expected tissue characteristic and sensed tissue characteristic for control of a surgical robot, such as for example robot 22 of system 30, in a pedicle screw hole drilling surgical task. It is to be understood that FIGS. 4-7 show the volume of the vertebra about the pedicle channel 44 in perspective view without cross-section; however, the trajectory for the surgical task is through the interior of the pedicle channel 44 in the interior of the vertebra. Accordingly, the drill bit 42 in the FIGS. is proceeding through the interior of the vertebra and not above the surface.

It is to be understood that this method can be applied with consequent modification to the technique. The tissue characteristic sensor 5 utilized in this example is a force sensor 5, the expected tissue characteristic is a force characteristic of expected tissue on the predefined trajectory, and the sensed tissue characteristic is a sensed force characteristic on the actual trajectory of the tool 3.

It is to be understood that tissue characteristics capable of being sensed other than by force characteristics are also suitable for use with the surgical system. For example, the system can utilize photonics and lasers to drill fine tracks in the bone or soft tissue, for example to implant strengthening rods or radioactive seeds. Sensors can be included to sense tissue distortion, for example, measured radiologically or by use of photonics.

A difference in the compared expected tissue characteristic 7 and the sensed tissue characteristic 13 can be used by the surgical system 30 to control the motion of the robot 22. For example, a drill bit 42 is used to drill a pedicle screw hole (surrounding drill bit) through a pedicle channel 44 of a vertebra 45. As the drill bit 42 proceeds through its pre-planned trajectory 46 to a destination 47 it encounters hard bone 48 then once through the hard bone 48 it encounter softer inner bone 50 that force sensor 5 senses as a resistive (anti-rotational) force of F1 on the drill bit 42.

As an example, the sensor 5 can be six axis force sensor 5 utilizing strain gauges mounted on a flexure to measure strains, and thus the forces, applied to the sensor 5. The sensor 5 is placed in the mechanical load path, so the loads are transferred through the flexure, where the strains are measured. Examples of the six axis sensed are described below. Such sensors are commercially available.

Alternatively, the sensor 5 can be a current sensor for a drill tool 3 of the robot 22. Current drawn by a drill tool 3 will be related to the resistive force on the drill bit 42. As the resistive force increases the current drawn will increase.

Other sensors 5 can be used, as an example can include pressure sensors 5. The type and location of the sensor 5 will depend upon the applicable tool, surgical task, and force to be sensed. Multiple sensors 5 may be used to derive a tissue characteristic from multiple tissue characteristics. Tissue characteristics may be sensed over time to derive a tissue characteristic.

As the drill bit 42 proceeds on its planned trajectory 46 the pedicle channel 44 narrows and it is possible that the actual trajectory of the drill bit 42 will result in the drill bit 42 encounter hard bone 48 at a wall 52 of the pedicle channel 44. This results in the force sensor 5 sensing a resistive force of F2 greater than F1. The sensed forces F1, F2 are transmitted back to the surgical system controller 11 on an ongoing basis in real-time and the controller 11 continuously compares the sensed forces against the expected forces. The controller 11 can stop the robot 22, or in more sophisticated applications the controller 11 can adjust the planned trajectory 46 to an adjusted trajectory 54 with a destination 55 to move away from the hard bone 48 and toward the soft bone 50 in the pedicle channel 44.

A six axis sensor 5 mentioned previously can provided some direction information as to where the force is being exerted. The surgical system can then adjust from the trajectory 46 to an adjusted trajectory 54 away from the force.

For a single axis sensor, such as the current sensor mentioned above the surgical system 30 may not know how to adjust the trajectory 46, the surgical system 30 may have to pull back the drill bit 42 slightly and take an initial correction. If less force is encountered then the surgical system 30 may continue on the adjusted trajectory 54 until further correction is required. If the same force is encountered, or a greater force is encountered at an earlier position, on the adjusted trajectory 54 then a readjusted trajectory can be attempted. Thus a desired adjusted trajectory can be iteratively obtained. Alternatively, a planned trajectory that favors a particular side of the pedicle channel 44 may be chosen. If the wall 52 of the pedicle channel 44 is encountered then an initial correction can be made in a direction away from the side that was favored.

Adjustment of a planned trajectory 46 based on sensed forces can be applied to many other surgical tasks, and tools.

Forces may be sensed in multiple degrees of freedom for example, an x, y and z axis. In a drill bit 42 application the x and z axis may be consider orthogonal lateral forces 60, 62, while the y axis may be a longitudinal force 64 along the drill bit axis. Three rotational forces 66, 68, 70 can include rotation about each of the x, y and z axis. As will be evident to those skilled in the art other coordinate systems may be used to define the forces being sensed.

Encountered forces may be sensed indications of tissues other than soft bone 50 and hard bone 48. For example, skin can present a different force characteristic from internal organs. Membranes may present different forces characteristics from the contents of the membranes. Anticipated force characteristics that match sensed force characteristics can be used to by the surgical system for automated control of the robot. For example, if a desired location is behind skin and two membranes, the sensed force can be used to count the punctures of the skin and the two membranes before an action is taken by the robot, such as acquiring a sample.

The principles described herein will be described primarily with respect to embodiments of surgical systems 30 providing a robot 22 under automated control with surgical tools 3 for use in performing a surgical task. In specific embodiments the robot 22 is image guided. It is to be recognized that some of the embodiments and functionality described herein do not require a robot, or an automated robot, or that the robot be image guided and such embodiments can be applied to guidance of surgical tools 3 outside of robot 22 under automated control.

Example interfaces of surgical systems with the OR and staff will be described. The surgical systems can be implemented utilizing robots 22 such as a master slave device modified to provide automated surgical procedures using robotic capabilities for following a predefined series of surgical steps/sequences to produce a desired surgical outcome. It is recognized that specific embodiments of the robots 22 described herein are referenced only as examples upon which to implement the guidance and other functionality described herein. Other robots 22 and tools 3 may be used to carry out the functionality described herein.

To enhance understanding of the principles described herein example surgical tasks will be outlined and an example description provided for robots 22 functioning as a single (or multiple) armed, image-guided system in the OR. Example tasks that can take advantage of tool guidance utilizing the principals described herein include pedicle screw hole drilling, needle insertion for the precision placement of medication such as spinal pain management, and biopsy, for example. Other example tasks that can be performed by an automated robot can include direct surgeon-in-the-loop (for directing a robot to perform a sequence of predefined surgical steps) and multiple armed applications for microsurgical and laparoscopic tasks, for example. It is recognized that the predefined surgical steps can be planned outside of the OR, inside of the OR, or a combination thereof.

For example, for many surgical procedures image guided capabilities can be added to a robot to accomplish automatic, image guided, drive-to-target applications. Pedicle screw insertion is an example of such applications and the majority of the remainder of this description will describe example embodiments with respect to pedicle screw insertion. Performance of defined surgical steps (collectively referred to as a surgical task) can be guided for example by images. Such images can be acquired using many well known techniques for surgical applications, such as fluoroscopic images, machine vision camera, and other imaging techniques that produce images of a patient and surgical tools (e.g. robot arms, OR environment, etc.) that can be interpreted by automated equipment, such as software executed in an computer forming part of an automated robot, in order to coordinate the positioning of the surgical tools with respect to the patient for the predefined surgical task(s). For example, the system can have the capability of accepting suitable images in DICOM format so that the system can be used with a fluoroscope when available. Also recognized is that a CT/Fluoro imaging system may be used to provide 3D images. Another example option is to use focused ultrasound scan (USS) as a means of tracking progress and providing ongoing information to the automated robot. USS information in some procedures may reduce the radiation exposure levels experienced from CT/Fluoro.

For some of the example surgical tasks described previously, the location of interest is internal and fluoroscopic, CT or MR image or other techniques are typically used for guidance information. In existing techniques surgeons may be required to interpret the guidance information and use anatomical cues and navigational tricks. In many cases surgeons perform the procedure ‘blind’, i.e. relying on the hands-on surgical abilities of the surgeon. If surgical precision (or other constraints) is critical for the success of the surgical task some embodiments of the surgical system can reduce time spent verifying the initial position and orientation of the tool to gain confidence that a straight, forward trajectory will reach the desired destination. Some embodiments of the surgical system can save precious time to verify anatomical tissue response and surgical precision issues during surgery.

Accordingly, some embodiments of the surgical system are particularly suitable to precise tool positioning at locations within the patient (as directed by image interpretation in view of the patient anatomy that is not directly visible to the surgeon). Other applicable surgical tasks can include surgical instrumentation or intervention including biopsy, excision or tissue destruction using a variety of chemical or electro-mechanical or temperature sources. Such tasks can be well suited to embodiments of the surgical system so that outcomes can be improved and surgical capabilities can be extended where they might otherwise be limited due to for example timing constraints, precision constraints, expertise/experience constraints. Some embodiments of the surgical system can be used to perform certain surgical tasks within a larger surgical procedure. Embodiments of the system can take a form to allow the robot to function like a fluoroscope, where the robot is rolled into the sterile field when it is needed for a particular task, and rolled out when it is finished. In some embodiments the surgical system is directly linked to an imaging system, for example a CT/fluoro machine which is used as needed, or based on predetermined timings (as part of the predefined surgical tasks) to acquire data to allow the system to control the robot to carry out specific precise surgical tasks based on a pre-planned set of actions.

The surgical system uses trajectory-following and destination-selection capabilities of a robot to address discrepancies, ‘close the loop’, between the destination seen in the image and the actual destination within the patient, as well as to deal with any encountered (e.g. not predefined) obstacles/hindrances/considerations during performance of the predefined surgical task. The surgeon is no longer performing a blind task, but rather is an intelligent connection between the information supplied by the image and the intended tool position defined in the physical world of the robot.

The surgeon is an intelligent connection in that the surgeon establishes the desired placement of the pedicle screws using the supplied image data. As surgical planning systems become more sophisticated it will be possible to interpret the image and determine from the image characteristics where the appropriate trajectory and destination. In current embodiments the surgeon performs this function.

Destination is the desired end point and trajectory is the direction to follow in reaching the reach the end point. A combination of the destination and trajectory provides a surgical path. There are many ways to specify the trajectory and destination, and thus the surgical path. For a straight line trajectory, the trajectory may be implied from a beginning point and an end point. A destination may be implied from a beginning point and a direction and a distance from the beginning point in the specified direction. Other ways in which a trajectory and destination may be specified will be evident to those skilled in the art. It is to be understood that a requirement for a trajectory and a destination does not require the actual trajectory and destination to be supplied, but rather information from which the trajectory and destination could be derived.

Thus for a surgical task to be performed by a surgical system utilizing an automated robot steps in an example can be:

1. Take one or more images (Imaging system)

2. Decide where to go (Surgeon)

3. Tell the robot where to go (Surgical system under instructions acquired from surgeon using robot planning interface)

4. Start the robot (Surgical system, authorized and monitored by surgeon)

5. Perform automated robotic task compensating for discrepancies using feedback (Robot under control of surgical system, monitored by surgeon)

6. End the task (Robot under control of surgical system, confirmed by surgeon)

Further example steps will now be described for carrying out a surgical task from preoperative planning to actual performance utilizing an embodiment of a surgical system with automated robot. First, a patient mounted localizer array within the images is registered with the system. Next, the robot is brought to the surgical field, and the patient localizer array is registered to the robot with the system. Registration is a process by which coordinates and distances in the image are matched to coordinates of the robot. As is known in the art this can be done in many different ways. Next, a tool of the robot is displayed together graphically on a monitor with the image, so that a surgeon can select an initial position, trajectory and final destination of the tool using the fused image (it is recognized that this definition of the predefined task(s)—e.g. travel from start position to end position—can be defined either in combination or separately with respect to inside/outside the OR). The surgical system transforms the starting point, trajectory and destination defined in the image to the robot coordinates and is able to automatically control the robot to move the tool to the destination. The precision of the movement is then dependent on the surgical system, including for example the mechanical design of the robot and the control precision, including any control software. The task may be virtually rehearsed if desired to confirm that the performed motion is what the surgeon intended (e.g. follows the surgical path predefined by the surgeon in a manner that is suitable to the surgical task). The surgical system provides interfaces to the surgeon to select the robotic motion, continually monitor the progress via the fused image, and have the ability to halt or modify motion of the robot at any time during performance of the surgical task(s). Embodiments of the surgical system also provides an interface to allow the surgeon to input safety parameters which allows the surgical system to function within specified safety zones, such as for example anatomical barriers, force tension barriers (an example of force feedback based on encountered tissue characteristics), and/or electromechanical recordings.

In an embodiment the surgical system 30 is configured to be stowed in the OR away from the sterile field until it is needed to effectively perform a given surgical task. Any patient preparation, dissection or exposure may be performed first by the surgeon in a traditional fashion. The robot 22 is bagged and rolled into the sterile field when it is time to perform the surgical task. The robot is configured for quick deployment by a nurse. For example, in the case of the pedicle screw drilling task, the robot is deployed after the spine is exposed and it is time to drill the holes.

The images used for image guidance are acquired at this phase of the operation. An example sequence of integration is as follows:

1. Bag and roll in the imager (for example a C-Arm fluoroscope)

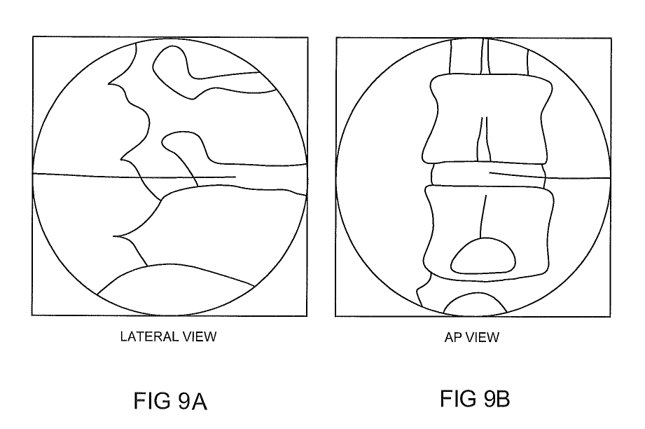

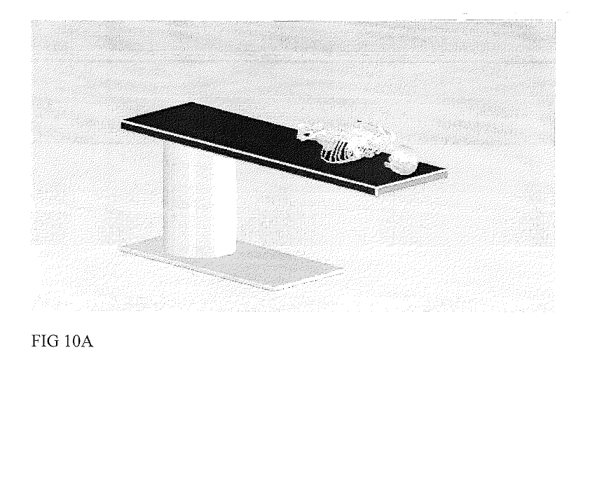

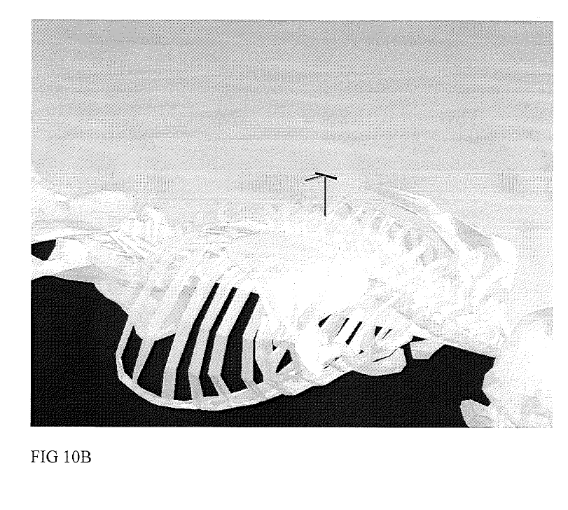

2. Mount the patient localizer array to the patient in a location where it will be visible in two images (FIG. 9A, 9B) to be taken in the subsequent steps. (FIGS. 10A, 10B)

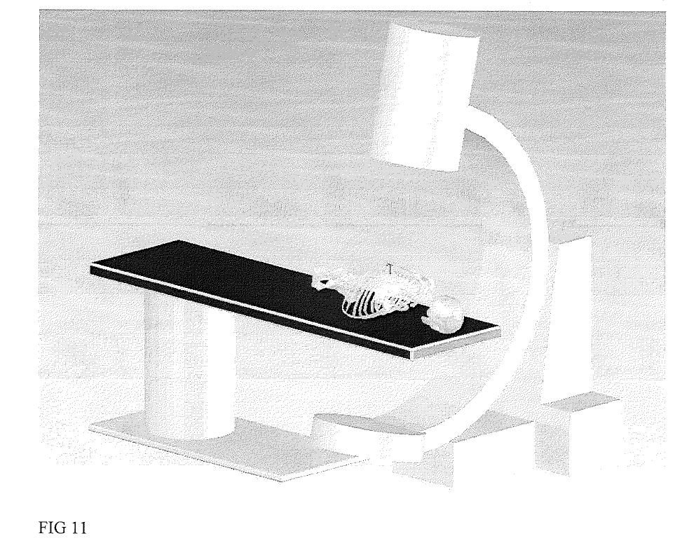

3. Position the imager such that the patient localizer array (PLA) is in the image as well as an anatomical feature of interest and take an image (FIG. 11)

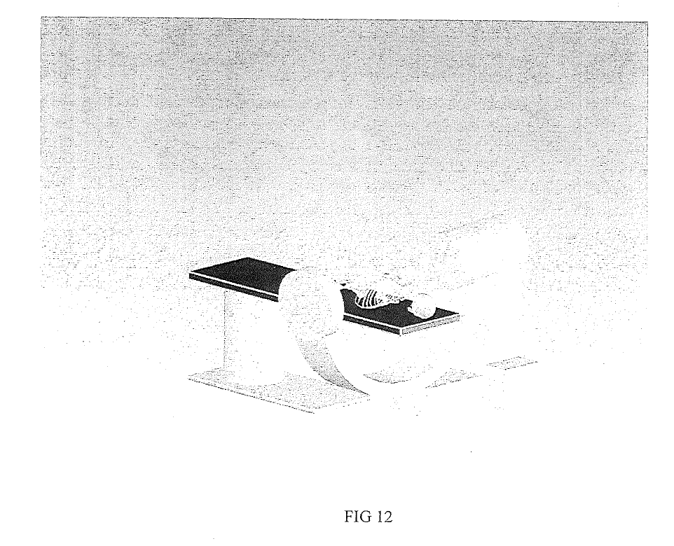

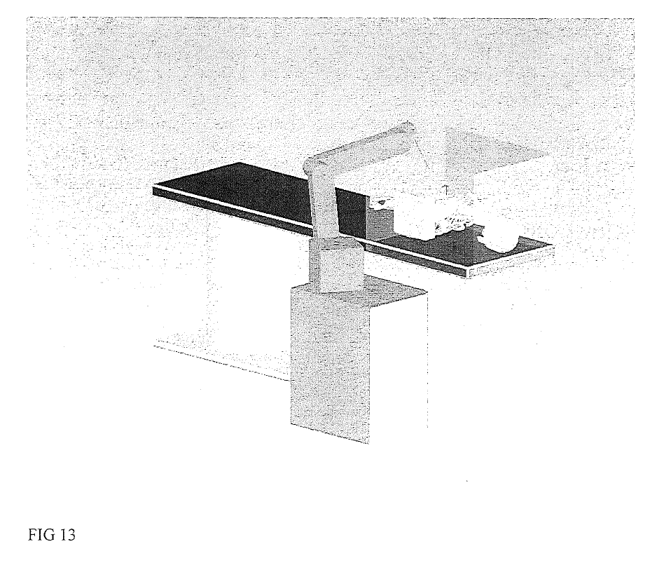

4. Take a second similar but orthogonal image (does not have to be 90 degrees from the first one but that is best). Again, the patient localizer array and the feature of interest must be in the imager field of view. (FIG. 12)

5. Remove the imager from the surgical site if it is in the way.

6. The robotic workstation will receive the data from the imager, register the patient localizer array (PLA) visible in the images.

The surgical system can be brought into the surgical field at this point, if it is not there already. Not all aspects of the surgical system are required to be in the surgical field, only those to be accessed by surgeon or other OR personnel in the surgical field and those that necessarily are required to be in the surgical field to perform there allotted task. The following example steps are anticipated for the preparation of the surgical system:

1. Bag using disposable plastic draping similar to a fluoroscope

2. Connect power, data, video

3. Connect to the imager workstation or other data port to load images

4. Power on and initialized

5. Fit with the procedure specific tool by a nurse/operator

6. Roll to a location beside the operating table, close to the surgical site

7. Anchor to the floor and/or mechanically fastened to the table (FIG. 13)

Note that all but the last two steps can be done before the surgical system is brought to the surgical site.

At this point, a tracking system will localize the patient mounted localizer array (PLA) and a robot end effector. Another aspect of the robot or a device localized to the robot can be used to localize the patient and the robot to use to track the robot as will be evident to those skilled in the art. A representation of an Aurora Tracking System from NDI is shown in FIG. 13, along with volume over which tools can be tracked (transparent volume in FIG. 13). From knowledge of these positions, the tool position can now be overlaid onto the images. Desired motions of the robotic system can now be programmed. To do this, the operator will:

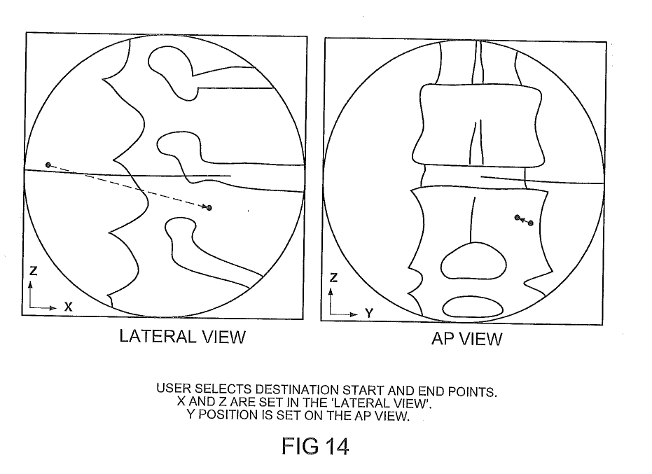

1. Identify start and final destination for the tool tip position, along with the desired orientation of the tool (FIG. 14)

2. Identify intermediate points if desired

3. Stay out (no go) zones may be selected at this time with the same input device. No go zones may be set based for example on location information as will be described again later in this description.

4. System moves robot to defined start position

Now, the tool can be automatically translated along the programmed trajectory via a hand controller deflection or single automove command.

During tool motion, the tracking system will monitor the positions of the array and the robot end effector to update the tool overlay and verify the trajectory in real time.

The PLA, which is visible in the fluoroscope images and also tracked by the tracking system in 3D space, provides the link between the patient location and the images used to guide the surgery. The PLA also allows for tracking of patient motion during the surgical task. Employing image guidance alone assumes that the anatomical target within the patient has a fixed relationship to the PLA from the point of where it is attached to the patient and images are taken, until the conclusion of the image guided portion of the operation.

The selected points are transformed by the surgical system under control of robotic control software into tool positional coordinates, which, when commanded to start by the surgeon, will be automatically followed by the robot. Possible limitations to the accuracy include for example the robot mechanical design and the quality of the image. The surgical system provides a display for the surgeon to monitor progress as tool motion is updated in real time and provides the surgeon with the ability to stop motion at any time. Until the surgeon intervenes the tool and surgical task are operating under the control of the surgical system.

Once the surgical system determines that the destination is reached, a second set of images can be taken for tool position verification. If the task is successful, the tool is removed from the surgical site with an automatic reverse motion. If the destination is not correct, a second trajectory and destination can be selected in the same way as the first trajectory was selected to adjust the tool position. Further holes can be drilled, or tissue samples obtained in the same manner. When the robotic task is complete, the tool is removed from the robot. The robot is disconnected, rolled out and de-bagged.

The surgical system must be compatible with standard procedures and processes of typical Operating Rooms (OR). The most important would be to maintain the sterility of the surgical field. Example methods include:
Sterilization of components in contact with the patient or near the surgical site,
Sterilization of components handled by surgical staff, and
Draping of non sterile components to form a barrier to the patient.

In order to be usable within the constraints of an existing operating room (OR) the size of those portions of the surgical system in the OR is kept to a minimum, as is the number of connecting cables.

Example interfaces between elements of the surgical system, external systems and an operator are:

1. User Interface (keyboard, mouse, display)

2. Tool Holder

3. Robotic System/Bed clamp

4. Imager/PLA

5. Image Data Interface to surgical system controller

6. Tracking System to PLA

7. Tracking System to Robot End Effector

8. Tracking System to surgical system controller (typically a computer)

9. Hand Controls and hand controllers for manual surgical operation

Example surgical system states and modes are summarized in Table 1.

TABLE 1
System States

| | |
|---|---|
| Off State | Arm unpowered |
| Home | Used to calibrate arm pose. (in the described |
| | embodiment arm joint encoders are incremental, |
| | so a reference position is used) |
| Limp | Used to position arm such that registration tool is |
| | in imaging volume. |
| Registration | Operator can select targets in an imported image |
| | to define the position of the PLA. This may be a |
| | semi-automated process to reduce the workload |
| | of the operator. |
| Trajectory Planning | Operator can select targets in an imported image |
| | to define the start and destination points in |
| | image space (where these defined the trajectory |
| | and destination as described previously). |
| Master/Slave | Tool tip moves under hand controller |
| | command(s) to facilitate operation. This |
| | trajectory may or may not be constrained to the |
| | pre-programmed trajectory as selected by the |
| | operator. |
| Automove | System can move along pre-programmed |
| | trajectory (e.g. the predefined surgical task(s)) is |
| | performed by the robot under control of the |
| | surgical system, such as straight-line motion |
| | (between two points) to a target in the surgical |
| | corridor as well as via predefined intermediate |
| | points/regions (waypoints) or obstacles (following |
| | a straight or otherwise curvilinear path). Another |
| | instance is motion from the current position to a |
| | user defined start position. This operation type is |
| | initiated by the surgeon using an initiate |
| | command to cause the sequence of steps to be |
| | performed as defined in the surgical task |
| | controlled by the surgical system. |

As described previously, the described embodiment of the surgical system can be moved in and out of the operating area and is used in the parts of the procedure that takes advantage of precise positioning of a tool or object. Example system functional capabilities include:

wheel up to an OR table and clamp to the side of the OR table in a straightforward manner

accept images from a medical imaging device

allow the operator to select features of interest in the images, such as registration targets and destination points

precisely holds and manipulates a tool along user-defined trajectories according to the operational precision capability of the surgical system, including for example capable travel distances, path shapes, speeds, error tolerances, and feedback considerations.

feedback considerations in movement of the robotic components, such as for example arms, tool-tips, shoulders, due to allowed forces and other considerations such as defined no-go zones

predictive capabilities using defined limits/constraints, such as for example maximum/minimum force, or tissue models, such as bone characteristics, flesh characteristics, or both constraints and models to facilitate recognition of encountered anatomical deviations from expected values, for example embodiments of the surgical system can recognize drop in resistive force encounter by the robot as an indicator for potential undesired fracture of bone, or alternatively, an increase in resistive force indicating an encounter of hard bone when desiring to follow a path through soft bone.

ability for switchable shoulders/arms/tool-tips for one or more (e.g. multi) armed configurations of the robot

telesurgery potential such that the surgical system is teleoperable, where it can be controlled and/or planned at the patient side or by a surgeon/interventionalist from a remote networked location, for example network communications with the surgical system can be based on Web-based control software

coordination of timing sequence for complex surgical tasks with the potential involvement of two or more surgeons, for example compatibility of two or more predefined surgical tasks implemented in sequence/tandem

A surgical system in accordance with one or more of the embodiments described herein can utilize incisions sufficient for entry of the tool only, thus result in reduction of incisions size over procedures that require the interaction of the surgeon during operation of the predefined task. This can include a reduced need to accommodate the ergonomic considerations of having direct interaction with the surgeon and the patient during operation of the surgical system.

Software, for example operating on a programmed controller, such as a computer, of the surgical system can facilitate implementation of the above-described capabilities, as performed by the surgical hardware of the surgical system.

To help the goal of quick deployment, the surgical system can be packaged to quickly roll-in and roll-out, anchor and fasten to the operating table, and connect with utilities such as power, data, and video. A further example configuration may incorporate a base of the robot into an operating table such that the robotic hardware components (e.g. shoulders) are attached directly to the table periphery, at locations based on the surgery/procedure to be performed. In this configuration, it is recognized that the robotic hardware components of the robot can move along the length of bed to be positioned in selected position(s) for imaging and/or performance of a predefined surgical task. The robot can also be designed to seamlessly connect to a selected standard imager or group of imagers.

The surgical system is broken into three major physical components, the arm(s) (e.g. surgical hardware) and associated base, the control electronics cabinet, and the workstation (e.g. containing the surgical system controller) and displays. Each component can be mounted on a base with wheels that can be moved by a nurse, however, the electronics cabinet may be shared among OR's and therefore may be mounted at a single central location, for example with cables routed to each OR.

The robotic arm is mounted to a base that contains features that permit attachment to the operating table and anchoring, or stabilizing to the floor. The volume of this component can be minimized at the operating table to allow access and space for attending surgeons and nurses. Tools can be manually or automatically attached and detached to the robotic arm by a nurse, as well as automatically recognized by the robotic controller as to the configuration of the coupled tools and arms. This is sometimes referred to in computer applications as plug and play capability.

The workstation component contains a large display, a hand controller for arm motion commands under direct surgeon control, a trajectory and destination selection device (such as a mouse and keyboard) and additional monitors for video and data displays. For example, IGAR can have three displays. One to display CT/Fluoro or USS imaging obtained. One to show the superimposed imaging and the surgical anatomy obtained from an outside camera (fused image) and showing the tracking markers to ensure visually to the surgeon that the system is operating correctly and a third to show the pre-planned action steps and what the next action is the robot going to do. Further displays can show other parameters such as robotic operational parameters (e.g. force sensing at the tip) and patient parameters (e.g. the temperature or pulse, etc.).

In any event, it is recognized that the surgical system under automated control is not a master-slave type setup (where all movements of the surgical hardware is under direct manipulation control of the surgeon), rather the surgical system allows for issuance of a command that causes the predefined surgical task to be automated as it performed under the control of the surgical system and under supervision (rather than direct manipulation) of the surgeon. It is also recognized that in appropriate situations (e.g. under emergency conditions or at preplanned surgeon hands-on interaction points) the surgeon can take control of the surgical system and perform the predefined surgical task and/or other surgical tasks manually, as desired.

The surgical robot can have a stop button or other interface which allows the surgeon to halt the performance of the predefined surgical task and a clutch system for the surgeon to enable and disable the robotic arm to use manually with the aid of the hand controller. In this case, it is recognized that the master-slave commands from the hand controller (as operated in real time by the surgeon) would be recognized as a substitute to the automatic operational steps included in the predefined surgical task.

A similar setup can be used in the planning mode to allow the surgeon to plan the set of movements and correct trajectory for robotic action. For example, in a test/planning procedure, the robot could be trained to learn the surgical task through interpreting the actual movements of the robotic hardware via the surgeon, when the surgical system is in the master-slave mode. In this case, the controller of the surgical system can be used to create the definitions for the predefined surgical task through monitoring and processing of the movements recorded in the master-slave mode. In this way, the master-slave mode could be used in the planning stage to help with the programming of the surgical system controller to create the definitions for the predefined surgical task.

As described herein the robotic arm for this system can be especially suited to automated microsurgical robotic tasks. The system as shown has a single arm which may be controlled telerobotically by a master hand controller for issuing commands to the robot to start the predefined surgical task(s). Robot manipulator having configuration other than the robotic arm illustrator herein may be used within the surgical system.

Referring to FIG. 14, the image guided capability (as coordinated by the programmable surgical system controller) enables the surgical robotic hardware to perform precise automated surgical tasks, according to implementation of a sequence of pre-defined steps for a desired surgical result, for example automated movement from a defined start point to a defined end point. For example, the imaging of the target site can be done with machine vision cameras. These can provide the images for the operator to register the tool in the image and select/predefine the trajectory for the robot to follow. The target sample is shown as a spine model representing a patient.

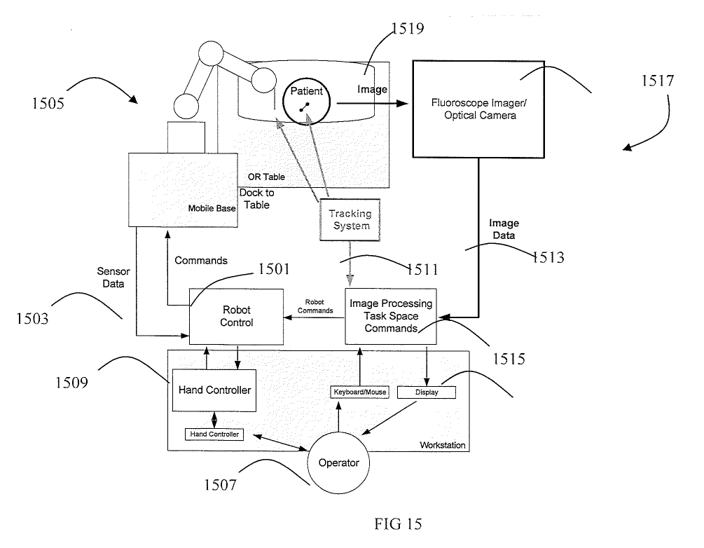

Referring to FIG. 15, in this embodiment a surgical system controller 1501 is a robot control obtaining input 1503 from sensors at robot 1505, the surgeon 1507 via a hand controller 1509 or other computer interface suitable for initiating or halting the performance of the predefined surgical tasks, and position feedback determined form interpretation of digital images by a combination of tracking system information 1511 with imager data 1513 as performed by an image processing task space command module 1515. It is recognized that the image processing task space command module 1515 could also be part of the robot control, as desired. It is also recognized that different functions of the robot control could be distributed throughout surgical system 1517. For example, additional intelligence could be built directly into the robot 1505 itself. Accordingly, it is to be understood that all functions of an surgical system controller 1501 can be integrated (for example on a single computer) alone or together with other components, such as the robot or image processor, and the functions of the surgical system controller 1501 can be distributed within the surgical system 1517.

In one embodiment, an operating bed or table 1519 can be associated with a robot with up to, for example, eight flexible robotic arms or manipulators in an operating room (OR) under control of the surgical system. Each of the arms can be releasably secured to a respective base station which can travel along a track system positioned on the perimeter of the table. It is noted that the base can be securely mounted to the tracking system, such that the base can be remotely controlled by the surgical system controllers to reposition the surgical hardware at various locations with respect to the anatomy of the patient on the table. The relative position and orientation of the surgical system hardware is monitored by the surgical system controller with respect to a common reference coordinate system, such as for example a room coordinate system, table coordinate system, patient coordinate system where patient position trackers are used. For example, the arms can have six degrees of freedom and can enable robotic surgery (as supervised by the surgeon) in cooperation with real time radiological evaluations by either, for example, CT, MRI or fluoroscopy imaging apparatus. Further, it is recognized that the selectable position capability of the base stations with respect to the table can add another motion degree-of-freedom to each arm that can be used by the surgical system controller to increase the workspace of the arm and/or maintain the distal arm position/orientation while moving the arm out of the way of other arms or another OR device, such as for example a fluoroscopic imager.

Sensors of the surgical system hardware provide position/orientation information of the base, arms, tool-tips as feedback to the surgical system controller, so as to help guide the surgical system hardware in view of the interpreted images during performance of the surgical task(s). The position tracking devices facilitate the surgical system to adjust to slight, for example micro movements of the patient during the performance of the surgical task. Micro movements may be, for example, small patient motions (breathing for instance), as opposed to gross motions, like standing up or rolling over. Depending on the task undertaken, the surgeon can determine the range of patient movement acceptable beyond which the system has to re-register its tool position in relation to predetermined landmark using a combination of tracking markers and CT/fluoro or USS imaging of internal organ landmarks, for example.

Position sensors can also provide data to the controller to facilitate automatic potential collision detection and avoidance between arms/tools, as well as to help in avoiding predefined no-go zones with respect to patient anatomy. Accordingly, the surgical system controller includes a data signal module for receiving/transmitting data to and from the arm, such as for example camera signals or position sensor signals, and a control signal module for transmitting control signals to actuated components of the arms, such as motors and camera operation, in performance of the predefined task. The control signal module also receives feedback signals from the actuated components of the arm, such as from force sensors.

Such force sensors can for example sense resistive force, such as anti-rotational resistance, being encountered by a drill bit as it moves through tissue in the body. Encountered forces can be compared against anticipated forces by the surgical system controller. Where there is a difference between the anticipated force and the encountered force then the surgical system controller can control the robot accordingly. For example, the robot can be stopped and an indication provided to the surgeon of the unexpected condition.

The surgical system controller is also coupled to a command module for receiving/confirming commands issued by the surgeon to initiate/halt the performance of the predefined surgical task. As well, the command module can also be used to provide feedback to the surgeon in terms of the progress of the surgical task, as well as to request for direction when parameters are encountered that are outside of the definitions of the predefined task, for example the occurrence or predicted occurrence of a bone fracture that was not anticipated in performance of the surgical task.

In general, it is recognized that the types of arms that are part of the surgical system hardware can be changed to suit the type of surgical procedure such as but not limited to laparoscopic, orthopedic, trauma, and microsurgery including neurosurgery and minimal access cardiac. It is recognized that the physical form/abilities and/or communications capability (with the controller) for each arm can be different as suits the intended surgical procedure for each specific arm/tool combination. For example, the surgical system can be configured with a common base for each category of procedures and the forearm hardware can be changed depending on the specific task to be performed. It is possible that in a single operation (e.g. including one or more surgical tasks), a number of different forearms may be needed to complete the whole operation. For example for drilling bone, a base and forearm is used capable of holding a drill and exerting the right amount of force, whereas for pain delivery or biopsy task a much smaller, thinned and more radio-opaque forearm may be used.

The arms and corresponding base stations preferably provide access to all parts of the patient in a single surgical procedure (i.e. predefined surgical task) as monitored by the surgeon, depending upon the particular selection of combined arms, instruments, base stations and their location with respect to the table. This combination can be used to provide a dynamically configurable surgical system suited to the planned surgical procedure on the patient. Configuration of the surgical system (either automatic, semi-automatic, and/or manual) can be facilitated by a configuration manager of the controller. Further, it is recognized that each arm has a proximal end that is coupled to the base station and a distal end for holding the surgical instruments. It is recognized that the arms can be articulated multi-segmented manipulators and that the base stations can be positioned independently of one another with respect to the table (e.g. one or more arms can be attached to one or more base stations). Further, articulation of each of the arms can be done independently through assigned control modules of the surgical system controllers. Various portions of the arms and the base stations are tracked for position and/or orientation in the coordinate system, as reported to the surgical system controller.

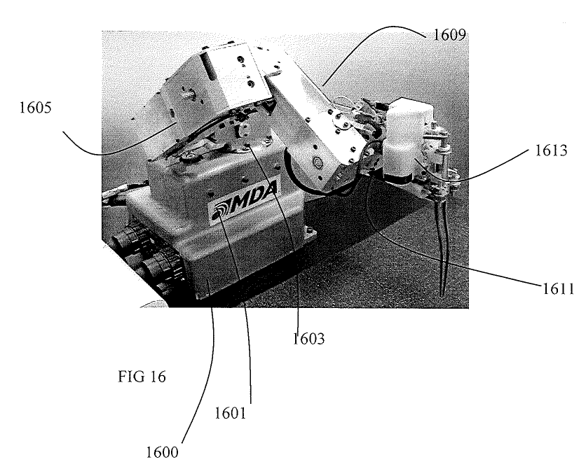

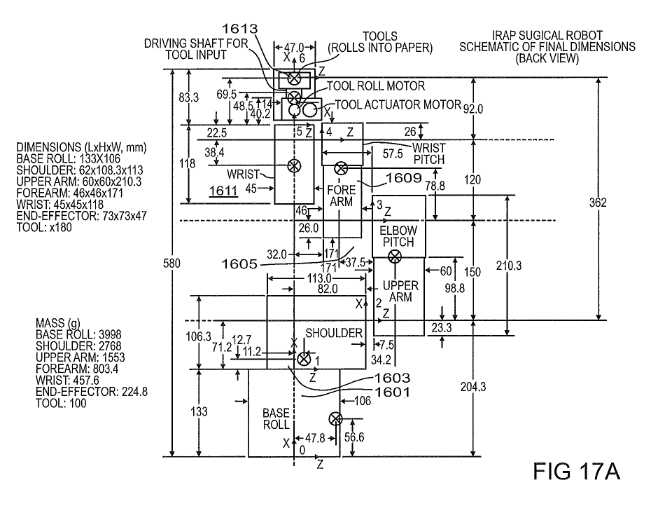

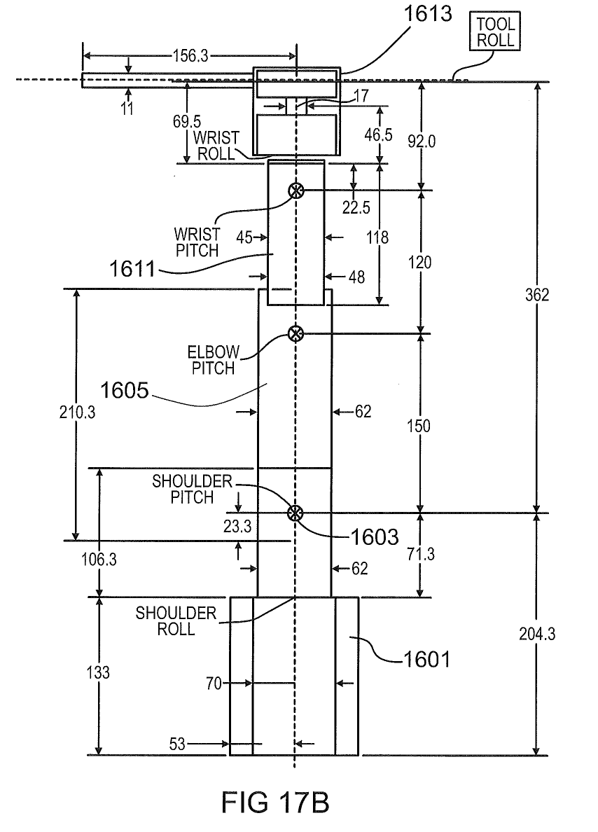

Referring to FIGS. 16, 17A and 17B an example robot has a base 1600 and a manipulator arm. The manipulator arm as shown has a plurality of segments: shoulder made up of a shoulder roll 1601 and shoulder pitch 1603, upper arm 1605, forearm 1609, wrist 1611 and an end-effector 1613. As will be understood by those skilled in the art, the segments are connected to form joints. Some joints have limited degrees of freedom to rotate about a single axis or multiple axes depending on the function of the segments as implied by the names used above.

The end effector 1613 provides an interface between the arm and any tools with a suitable corresponding interface. The end effector 1613 allows for manipulation of the tool, such as rotation or actuation of a tool function. It may also contain an electrical interface to connect to any sensors on the tool, actuate any electrical devices on the tool or identify the tool.

Solely for the purpose of context and not to limit the breadth of possible robot configurations, example dimensions for the robot illustrated in FIGS. 16, 17A and 17B are in length by width by height in millimeters are base 1601 133×(variable)×106, Shoulder 1603 62×108×113, upper arm 1605 60×60×210, forearm 1609 46×46×171, wrist 1611 73×73×47 and end effector 1613 45×45×118.

The surgical system can recognize what forearm is attached, thus adapting its maneuverability and functionality to the series of tasks which can be achieved with the specific forearm. The system can be adapted to automated tool change by disconnection and connection of tools with the end effector to complete a set of surgical tasks in sequence that require different tools.

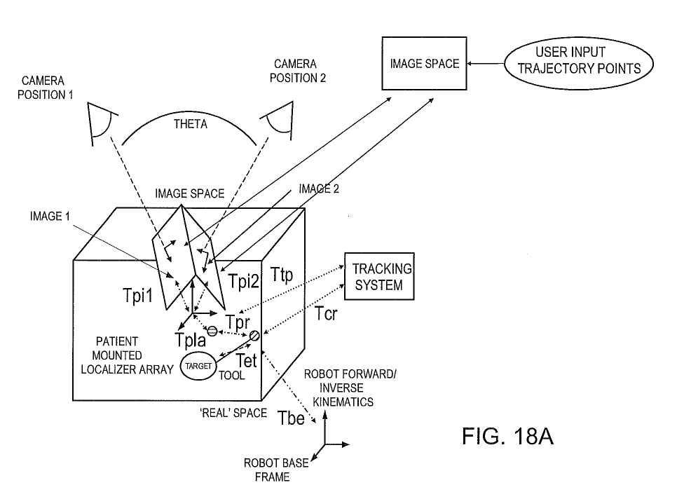

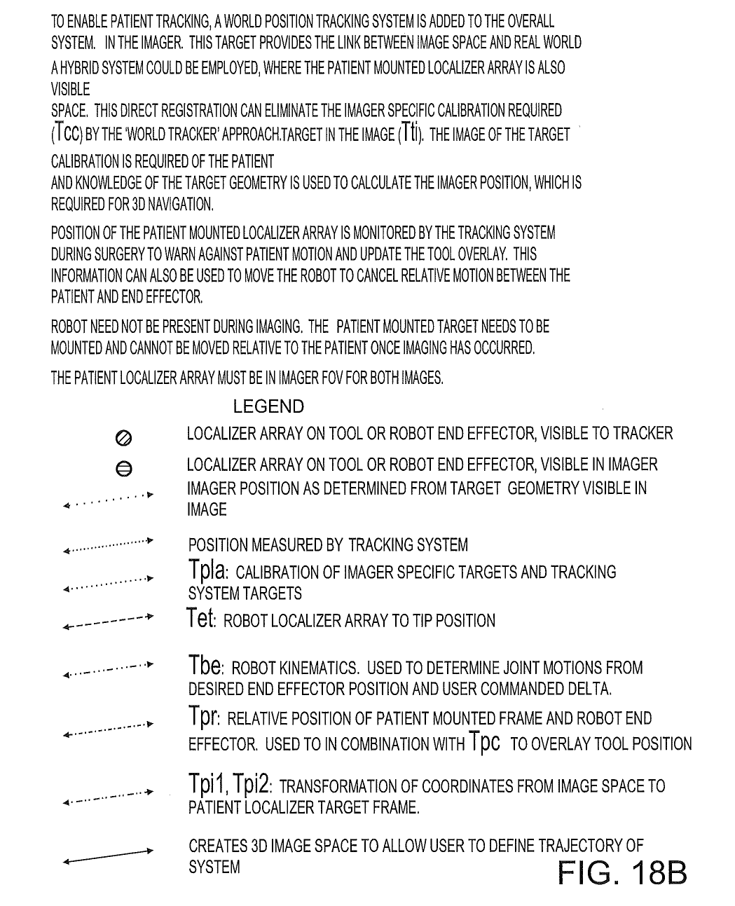

Referring to FIG. 18, in order to drive the robot to features of interest in the images, a link between the robot coordinate frame and the image coordinates is established. Further, the two images are combined in order to establish the position of features in three dimensions. The relative camera position for each image is not known from the imager.

The position of the patient is monitored during the operation so that motion of the patient can be identified.

In order to guide the tool in 3D space, based on fluoroscope images, a patient mounted localizer array (PLA) is used as mentioned previously. This provides a reference frame to located features in the fluoroscope images. The same feature is located in two different (non co-planar) images to locate these points in 3D space relative to the PLA. The robot is located relative to the PLA via an external Tracking System. This locates the PLA and the robot end effector (via embedded targets). The relative position of these features allows the robot held tool to be overlaid on the fluoroscope images, and the robot position to be guided by operator inputs on the images.

An example registration process can involve:

PLA exists in both images (2D) and real space (3D).

Features of interest, identified by the user in the 2D images. In order to position these in 3D space, the position of the features of interest, relative to the PLA, is determined in each image.

The PLA is used to link the positions of features in the images to relative to the robot end effector.

The robotic system does not need to be in place when the images are taken. The PLA needs to be in place, and cannot move in order to guide the robotic system via the acquired images.

To enable patient tracking, a world position tracking system is added to the overall system.

A hybrid system could be employed, where the patient mounted localizer array is also visible in the imager. This target provides the link between image space and real world space. This direct registration can eliminate the imager specific calibration required (Tcc) by the ‘world tracker’ approach.

Calibration is performed of the patient target in the image (Tti). The image of the target and knowledge of the target geometry is used to calculate the imager position, which is used for 3D navigation.

Position of the patient mounted localizer array is monitored by the tracking system during surgery to warn against patient motion and update the tool overlay. This information can also be used to move the robot to cancel relative motion between the patient and end effector.

The robot need not be present during imaging. The patient mounted target is mounted and is kept stable relative to the patient once imaging has occurred.

The patient localizer array is kept in imager field of view for both images.

Localizer array on tool or robot end effector is kept visible to tracker

Localizer array on tool or robot end effector is kept visible in imager

Imager position as determined from target geometry visible in image.

Position Measured by tracking system.

Tpla: Calibration of imager specific targets and tracking system targets.

Tet: Robot localizer array to tip position.

Tbe: Robot kinematics. Used to determine joint motions from desired end effector position and user commanded delta.

Tpr: Relative position of patient mounted frame and robot end effector. Used to in combination with Tpc to overlay tool position.

Tpi1, Tpi2: Transformation of coordinates from image space to patient localizer target frame.

Creates 3D image space to allow user to define trajectory of system.

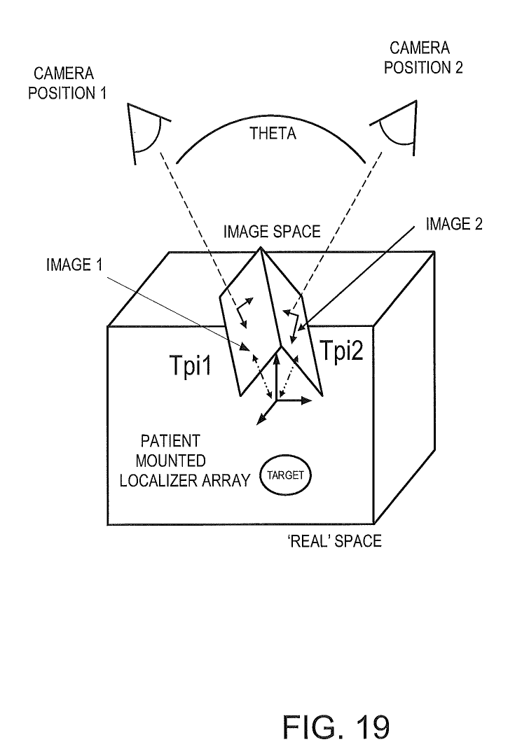

Referring to FIG. 19, the patient mounted localizer array (PLA) is attached to the patient. The imager (fluoroscope or demo camera) is then used to take two images of the patient with the PLA. The PLA position is located in each image by the system. Using knowledge of the target geometry, the camera positions are determined, allowing for localization of the PLA in space.

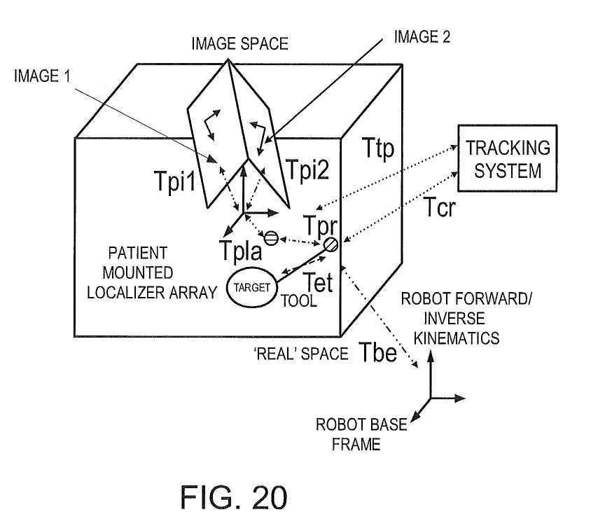

Referring to FIG. 20, after the initial imaging step, the imager can be removed from the area. The robotic system is brought to the surgical site, along with a tracking system that will localize the PLA and the robotic system.

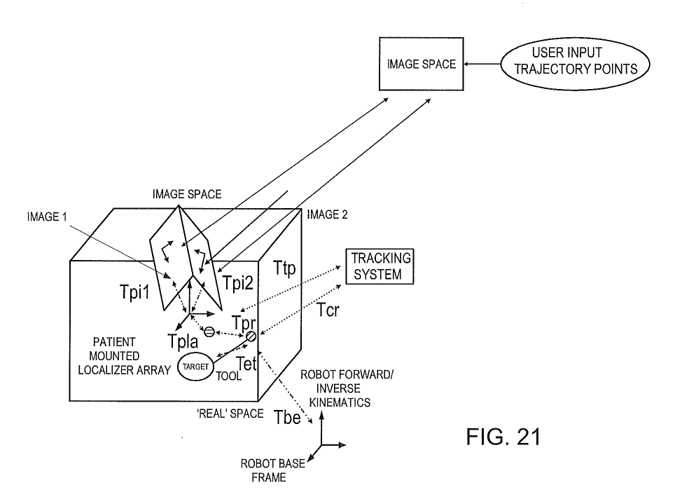

Referring to FIG. 21, the robotic system can then be guided, relative to the PLA, to sites identified by the operator in the images.

As the fluoroscopic imager produces an image that is a projection of objects that are between the head and the imager sensor, a point that is selected in one image represents a line of possible points in 3D space. The purpose of the second image is to locate the position of the point of interest along the line.

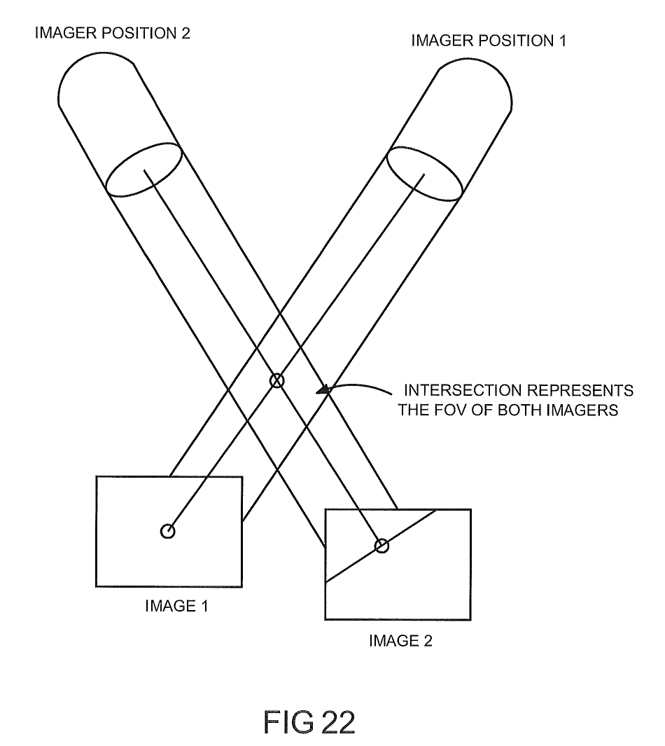

Referring to FIG. 22, the point selected in Image 1 (the red point), represents a locus of possible points represented by the red line. Selecting a point in Image 2 (the dot in image 2), also represents a locus of possible points represented by the green line. The intersection of these points represents the desired point. Once the first point is selected, the range of possible points in the second image can be limited to possible valid point (along the diagonal line extending from the centre of image 1 to imager position 1).

The relative positions of the imager need to be known when image 1 and image 2 are taken. This can be calculated based on the registration of the PLA in these images.

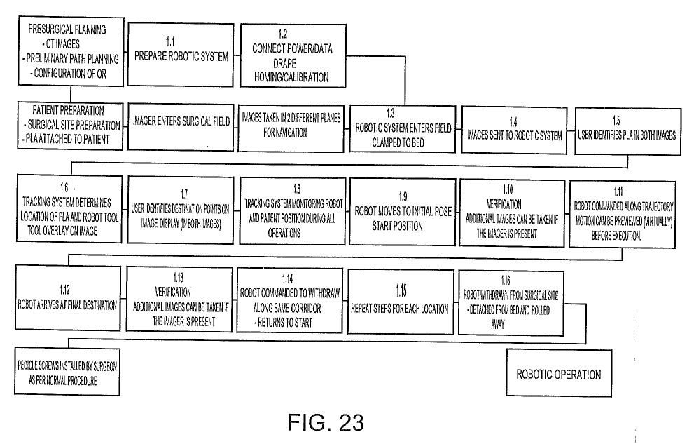

Referring to FIG. 23, an example functional flow of the system is illustrated in block form. Additional detail of selected example steps is given in the following sections.

As the robotic system in the described embodiment does not have absolute position encoders, each joint is required to find a home position in order for the system to understand its pose. This operation is done away from the surgical field as part of the preparation procedures. The system can be draped at the same time. Absolute position encoders could be utilized, if desired.

Trajectory planning is performed in the example described by the operator via the workstation interface. A start and end point are defined, along with any desired way points via an input device, such as a mouse or keyboard, on the acquired images. The motion of the robotic system can be simulated on the screen before the system is commanded to move so that the user can verify the intended motion of the system.

The robotic system can advance the tool along the planned trajectory in two different modes: Master/Slave—the operator can control the position of the tool along the defined trajectory; or Automove—the operator can select a speed at which the tool will be moved automatically along the defined trajectory from a start position to a defined destination. This may include a limited number of way points, if desired.

During the homing/calibration procedure the performance of the system is monitored. Any errors or out of tolerance behavior are identified at this time.

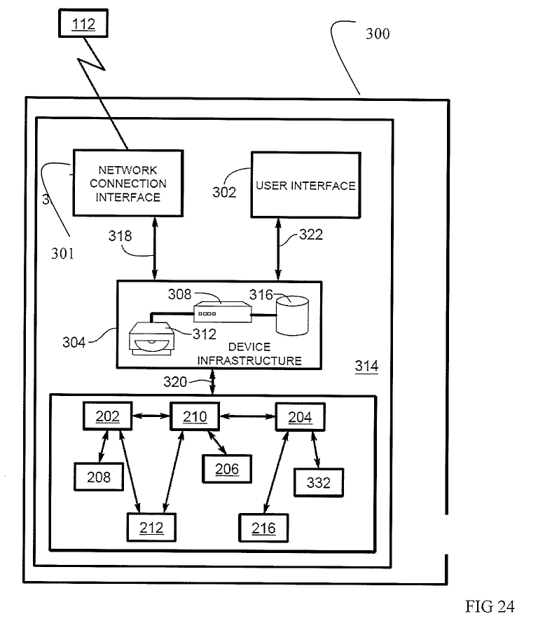

Referring to FIG. 24, shown is further example embodiment of surgical system utilizing a computer 314 that has control module, which computer and control module act together as controller 300 for controlling a robotic system 112. The computer 314 includes a network connection interface 301, such as a wireless transceiver or a wired network interface card or a modem, coupled via connection 318 to a device infrastructure 304. The connection interface 300 is connectable during operation of the surgical system. The interface 300 supports the transmission of data/signaling in messages between the computer 314 and the robotic system 112. The computer 314 also has a user interface 302, coupled to the device infrastructure 304 by connection 322, to interact with an operator (e.g. surgeon). The user interface 302 includes one or more user input devices such as but not limited to a QWERTY keyboard, a keypad, a track wheel, a stylus, a mouse, a microphone and the user output device such as an LCD screen display and/or a speaker. If the screen is touch sensitive, the display can also be used as the user input device as controlled by the device infrastructure 304. The user interface 302 is employed by the operator of the computer 314 (e.g. work station) to coordinate messages for control of the robotic system 112.

Operation of the computer 314 is enabled by the device infrastructure 304. The device infrastructure 304 includes a computer processor 308 and the associated memory module 316. The computer processor 308 manipulates the operation of the network interface 300 and the user interface 302 by executing related instructions, which are provided by an operating system and a control module embodied in software located, for example, in the memory module 316. It is recognized that the network interface 300 could simply be a direct interface 300 to the robotic system 112 such that commands could be issued directly to the robotic system 112 without requiring the commands to go through a network. Further, it is recognized that the device infrastructure 304 can include a computer readable storage medium 312 coupled to the processor 308 for providing instructions to the processor and/or to load/update the control module in the memory module 316. The computer readable medium 312 can include hardware and/or software such as, by way of example only, magnetic disks, magnetic tape, optically readable medium such as CD/DVD ROMS, and memory cards. In each case, the computer readable medium 312 may take the form of a small disk, floppy diskette, cassette, hard disk drive, solid-state memory card, or RAM provided in the memory module 310. It should be noted that the above listed example computer readable mediums 312 can be used either alone or in combination.

It is recognized that the control module, or portions thereof, could be installed and executed on computer 314, which could have various managers 202,204,208,210,212 installed and in communication with themselves, the robotic system 112 and/or the surgeon. The control module uses the user interface 302 for providing operator input to the robotic system 112 via the performance of the surgical tasks as facilitated by associated managers/modules 202,204,208,210,212,216 which could be for example configuration, communication, command, image interpretation, and other modules, as desired, to facilitate the performance of the predefined surgical task. For example, a communication manager provides for communication of data signals to/from the data manager and communication of control signals to/from a control manager. The database manager provides for such as but not limited to persistence and access of image data to/from an image database, data related to the functioning/set-up of various elements of the robotic system 112, for example arms, base station, actuators, and various position/orientation sensor data, and for providing data as needed to a position and orientation manager. A control manager, in cooperation with the control module and position/orientation information, provides for monitoring the operation of the arms, base stations, actuators, imaging equipment (for example a camera), and tools. The position/orientation manager is responsible for such as but not limited to receiving sensor data from the data manager for calculating the position and orientation of the respective arm components, tools, base stations, patient, and tabletop. The calculated position/orientation information is made available to such as but not limited to the performance progress of the predefined surgical task(s), the display manager, and the control manager. The configuration manager provides for such as but not limited to dynamic configuration of selected arms, base stations, the controller 300 (for example programming of parameters used to defined the predefined task), and a tabletop comprising the desired robotic system 112 setup for a particular surgical procedure. The dynamic configuration can be automatic, semi-automatic, and/or manual operator intervention. The display manager of the computer 314 coordinates/renders the calculated position/orientation information and the patient/tool images on the display of the user interface 302, for monitoring by the operator. For automated operation of the robotic system 112 surgical information displayed on the display (e.g. including real-time images of the patient and the tool) is not required to be interpreted by the surgeon in order to facilitate the performance of the predefined surgical task, rather the displayed information can be viewed by the surgeon in order to monitor the progression of the predefined surgical task that is controlled by the control module in view of sensor information and interpreted image data.

In view of the above, it is also recognized that further capabilities of the controller 200 can include: pre-programmed activity of the planned surgery (i.e. surgical steps and required arms and instruments combinations); pre-programmed safety protocols for controlling the arms in the surgical environment; and necessary instruments for the surgery as well as instruments suitable for selected arm types, as facilitated by the configuration manager. It is also recognized that the controller 300 can be programmed (using the predefined surgical task) to inhibit movement of the arms and associated instruments into predefined no-go zones with respect to internal regions of the patient and external regions of the OR. The controller 300 can facilitate the control of the arms and base stations to perform a variety of robotic surgeries in neurology, orthopedic surgery, general surgery, urology, cardiovascular and plastic surgery, for example. The controller 300 can also facilitate tele-robotic remote surgery by the surgeon from a remote distance.

In numerous places throughout this description the text refers to an example. Such examples are made for the purpose of assisting in the comprehension of what is being described. Such examples are without limitation to the description and other examples beyond those specifically listed can apply.

Various example features and functionality have been described with reference to example embodiments. It is understood that features and functionality from one embodiment may be utilized in other embodiments as desired and the context permits.

Although the present application has been described with reference to illustrative embodiments, it is to be understood that the present disclosure is not limited to these precise embodiments, and that various changes and modifications may be effected therein by one skilled in the art.

What is claimed is:

1. A surgical system for use with a surgical tool and a tissue characteristic sensor associated with the surgical tool, the system comprising:
a) an expected tissue characteristic for tissue on a predefined trajectory of the tool in a patient,
b) a controller to receive a sensed tissue characteristic from the tissue characteristic sensor, such sensed tissue characteristic associated with an actual trajectory of the tool, wherein the controller compares the expected tissue characteristic for the expected location with the sensed tissue characteristic for the actual trajectory.

2. The system of claim 1 further comprising a display displaying information to an operator of the tool based on the compared expected tissue characteristic and sensed tissue characteristic.

3. The system of claim 1 wherein the tool is operated by an operator through manual operation of the tool.

4. The system of claim 1 further comprising a robot for manipulating the tool, wherein the tool is operated by the operator through the operator manually operating the robot.

5. The system of claim 1 further comprising the tissue characteristic sensor.

6. The system of claim 1 further comprising the surgical tool.

7. The system of claim 1 further comprising a robot for manipulating the tool under control of the controller, which control is based on the compared expected tissue characteristic and sensed tissue characteristic.

8. The system of claim 1 wherein the tissue characteristic sensor is a force sensor, the expected tissue characteristic is a force characteristic of expected tissue on the predefined trajectory, and the sensed tissue characteristic is a sensed force characteristic on the actual trajectory of the tool.

9. The system of claim 7 further comprising means for an operator to monitor robot performance while under the control of the controller.

10. The system of claim 7 further comprising means for an operator to assume control away from the controller of the manipulation of the tool.

11. A method of using a surgical system, the method comprising:
a) receiving at a controller within the surgical system from a tissue characteristic sensor a sensed tissue characteristic associated with an actual trajectory of a surgical tool,
b) comparing within the controller the expected tissue characteristic for the expected location with the sensed tissue characteristic for the actual trajectory.

12. The method of claim 11 further comprising displaying information on a display to an operator of the tool based on the compared expected tissue characteristic and sensed tissue characteristic.

13. The method of claim 11 wherein the tool is operated by an operator through manual operation of the tool.

14. The method of claim 11 wherein the tool is operated by the operator through the operator manually operating the robot for manipulating the tool.

15. The method of claim 11 further comprising sensing the tissue characteristic through the tissue characteristic sensor.

16. The method of claim 11 further comprising controlling a robot under control of the controller to manipulate the tool, which control is based on the compared expected tissue characteristic and sensed tissue characteristic.

17. The method of claim 11 wherein the tissue characteristic sensor is a force sensor, the expected tissue characteristic is a force characteristic of expected tissue on the predefined trajectory, and the sensed tissue characteristic is a sensed force characteristic on the actual trajectory of the tool.
